SEQUENCE LISTING (4 sequences)

SEQ ID NO: 1 - DNA, 3822 bp, Escherichia coli
  First DNA Sequence
atgttcgttttcctggttctgctgccgctggttagcagccaatgcgtgaatctgaccacccgcacccaactgccgccggcgtacaccaacagcttcacccgtggtgtttactatccggacaaagtttttcgtagcagcgtgctgcacagcacccaggacctgttcctgccgttctttagcaacgttacctggttccacgcgatccacgtgagcggcaccaacggcaccaagcgtttcgacaacccggtgctgccgtttaacgatggtgtttacttcgcgagcaccgagaagagcaacatcattcgtggttggatttttggcaccaccctggacagcaaaacccagagcctgctgatcgttaacaacgcgaccaacgtggttattaaggtgtgcgagttccaattttgcaacgatccgttcctgggcgtttactatcacaagaacaacaaaagctggatggagagcgaatttcgtgtttatagcagcgcgaacaactgcacctttgagtacgtgagccagccgttcctgatggacctggaaggcaagcaaggcaacttcaaaaacctgcgtgagttcgtgttcaagaacattgatggttacttcaaaatctacagcaagcacaccccgatcaacctggttcgtgacctgccgcagggttttagcgcgctggagccgctggttgacctgccgatcggtattaacatcacccgttttcaaaccctgctggcgctgcaccgtagctacctgacgccgggtgacagcagcagcggttggaccgctggtgctgcggcgtactatgttggttacctgcaaccgcgtaccttcctgctgaaatacaacgaaaacggcaccatcaccgatgcggttgattgcgcgctggacccgctgagcgaaaccaagtgcaccctgaagagcttcaccgtggagaagggtatttatcagaccagcaacttccgtgtgcaaccgaccgaaagcattgttcgttttccgaacatcaccaacctgtgcccgtttggcgaggttttcaacgcgacccgtttcgcgagcgtgtatgcgtggaaccgtaaacgtatcagcaactgcgttgcggactatagcgtgctgtacaacagcgcgagcttcagcacctttaagtgctatggtgtgagcccgaccaaactgaacgatctgtgctttaccaacgtttacgcggatagcttcgtgattcgtggcgacgaggttcgtcagatcgcgccgggtcaaaccggcaagattgcggactacaactataaactgccggacgatttcaccggctgcgttatcgcgtggaacagcaacaacctggatagcaaagtgggtggcaactacaactatctgtaccgtctgtttcgtaagagcaacctgaaaccgttcgagcgtgacattagcaccgaaatctaccaggcgggtagcaccccgtgcaacggtgttgagggctttaactgctatttcccgctgcaaagctacggtttccaaccgaccaacggtgttggttaccagccgtaccgtgtggttgtgctgagctttgaactgctgcacgcgccggcgaccgtgtgcggcccgaagaagagcaccaacctggtgaagaacaaatgcgtgaacttcaactttaacggcctgaccggcaccggcgtgctgaccgagagcaacaagaaattcctgccgtttcagcaattcggtcgtgacatcgcggataccaccgatgcggtgcgtgacccgcagaccctggagatcctggacatcaccccgtgcagcttcggtggcgttagcgtgatcacgccgggcaccaacaccagcaaccaggttgcggtgctgtatcaagacgttaactgcaccgaagttccggtggcgattcacgcggatcagctgaccccgacctggcgtgtgtacagcaccggcagcaacgttttccaaacccgtgcgggttgcctgattggtgcggagcacgtgaacaacagctatgaatgcgacattccgatcggtgcgggcatttgcgcgagctaccagacccaaaccaacagcccgcgtcgtgcgcgtagcgttgcgagccagagcatcattgcgtatacgatgagcctgggtgcggaaaacagcgtggcgtacagcaacaacagcattgcgatcccgaccaacttcaccattagcgtgaccaccgagatcctgccggttagcatgaccaaaaccagcgtggactgcaccatgtatatctgcggcgatagcaccgaatgcagcaacctgctgctgcaatacggtagcttttgcacccaactgaaccgtgcgctgaccggcattgcggttgagcaggataaaaacacccaagaagttttcgcgcaggtgaagcaaatttacaaaaccccgccgatcaaggactttggtggcttcaactttagccagatcctgccggacccgagcaagccgagcaaacgtagctttattgaggacctgctgttcaacaaggttaccctggcggatgcgggtttcatcaaacagtatggtgattgcctgggcgacattgcggcgcgtgacctgatctgcgcgcaaaagtttaacggcctgaccgtgctgccgccgctgctgaccgatgaaatgattgcgcagtacaccagcgcgctgctggcgggcaccattaccagcggttggacctttggtgcgggtgcggcgctgcaaatcccgtttgcgatgcaaatggcgtatcgtttcaacggtattggcgttacccagaacgtgctgtacgagaaccagaagctgatcgcgaaccaatttaacagcgcgattggtaaaatccaggatagcctgagcagcaccgcgagcgcgctgggcaaactgcaagatgttgtgaaccagaacgcgcaagcgctgaacaccctggttaagcagctgagcagcaacttcggtgcgattagcagcgtgctgaacgacatcctgagccgtctggacaaagttgaggcggaagtgcaaattgaccgtctgatcaccggccgtctgcaaagcctgcaaacctatgtgacccagcaactgattcgtgcggcggaaattcgtgcgagcgcgaacctggcggcgaccaagatgagcgagtgcgttctgggtcagagcaagcgtgtggacttttgcggtaaaggctatcacctgatgagcttcccgcagagcgcgccgcacggcgttgtgtttctgcacgttacctacgtgccggcgcaagaaaagaactttaccaccgcgccggcgatctgccatgatggtaaagcgcactttccgcgtgaaggtgttttcgtgagcaacggcacccactggtttgttacccagcgtaacttctacgagccgcaaatcattaccaccgacaacaccttcgtgagcggtaactgcgatgttgtgattggcatcgttaacaacaccgtgtatgacccgctgcaaccggagctggacagcttcaaagaggaactggataaatacttcaagaaccacaccagcccggacgttgatctgggtgacattagcggcatcaacgcgagcgttgtgaacattcaaaaggagatcgaccgtctgaacgaagtggcgaaaaacctgaacgaaagcctgatcgacctgcaagagctgggcaagtatgaacaatacattaaatggccgtggtacatttggctgggtttcatcgcgggcctgattgcgatcgttatggtgaccatcatgctgtgctgcatgaccagctgttgcagctgcctgaagggttgttgcagctgcggcagctgctgcaagtttgatgaggacgatagcgagccggttctgaaaggcgtgaagctgcattacacctaa

SEQ ID NO: 2 - DNA, 35 bp, Artificial Sequence
  Second DNA Sequence
ccctagaaagatagtctgcgtaaaattgacgcatg

SEQ ID NO: 3 - DNA, 63 bp, Artificial Sequence
  Third DNA Sequence
catgcgtcaattttacgcatgattatctttaacgtacgtcacaatatgattatctttctaggg

SEQ ID NO: 4 - protein, 12 aa, Artificial Sequence
  linker
GGSGGSGGSGGS

(19) United States
(12) Patent Application Publication
(10) Pub. No.: US 2022/0412959 A1
(43) Pub. Date: Dec. 29, 2022

(54) METHOD AND PLATFORM FOR ENHANCING DETECTION ACTIVITY OF INTERACTION BETWEEN SPIKE PROTEIN RECEPTOR BINDING DOMAIN OF CORONAVIRUS FROM SPECIMEN AND HUMAN ANGIOTENSIN-CONVERTING ENZYME II

(71) Applicant: National Tsing Hua University, Hsinchu (TW)

(72) Inventors: Hui-Ching Wang, Hsinchu (TW); Tian-Neng Li, Hsinchu (TW)

(21) Appl. No.: 17/504,644

(22) Filed: Oct. 19, 2021

(30) Foreign Application Priority Data
Jun. 23, 2021 (TW) 110122937

(51) Int. Cl. G01N 33/50; G01N 33/569

(52) U.S. Cl. CPC G01N 33/5041; G01N 33/56983; G01N 2500/02; G01N 2333/165

(57) ABSTRACT

The present disclosure provides a method and a platform for enhancing detection activity of an interaction between a spike protein receptor binding domain of coronavirus from a specimen and a human angiotensin-converting enzyme II. The method and the platform of the present disclosure use a cleavable luciferase as a report test for the combination of the spike protein receptor binding domain of coronavirus (such as novel coronavirus) and angiotensin-converting enzyme II. Screening is carried out at the cellular level. The strength of the drug's influence on the interaction between the two molecules can be judged by the strength of the luminescence signal. The detection time can be completed within 20 minutes.

### CROSS-REFERENCE TO RELATED APPLICATION

This application claims priority of Taiwan patent application No. 110122937, filed on Jun. 23, 2021, the content of which is incorporated herein in its entirety by reference.

### BACKGROUND OF THE INVENTION

### 1. Field of the Invention

The present invention relates to a method and a platform for enhancing detection activity of an interaction between a spike protein receptor binding domain of coronavirus from a specimen and a human angiotensin-converting enzyme II (hACE2).

### 2. The Prior Art

The subfamily Orthocoronavirinae, commonly known as coronavirus, is a type of zoonotic single-stranded RNA virus that spreads between animals and humans. Coronavirus can infect mammals and birds and cause digestive tract diseases in cattle and pigs or upper respiratory tract diseases in chickens. Common in nature, there are seven types of coronaviruses that are known to infect humans. They can cause respiratory infections in humans, causing common colds, even Middle East respiratory syndrome (MERS), severe acute respiratory syndrome (SARS) and 2019 novel coronavirus disease (COVID-19) and other serious diseases.

The novel coronavirus disease (COVID-19) is an ongoing pandemic caused by severe acute respiratory syndrome coronavirus 2 (SARS-CoV2). The disease was first reported in November 2019 in China, and soon spread rapidly across countries. By March 2020, the World Health Organization (WHO) declared the COVID-19 a pandemic disease. Since then the COVID-19 pandemic has devastating social and economic consequences for the globe. By September 2020, there have been over 30 million confirmed cases globally, including 1 million deaths, reported to WHO. The control of the disease outbreak is not successful at this stage, as daily confirmed new cases continue to break historical record. Increasing number of daily confirmed cases is observed in South-East Asia, and a second wave COVID-19 outbreak is spreading across the Europe, Eastern Mediterranean, and Western Pacific. How to provide effective treatment and individual isolation pose a lot of pressure for the health system.

SARS-CoV2 uses the same cell entry receptor as SARS-CoV, the angiotensin-converting enzyme II (ACE2). The crystal structure of the receptor-binding domain (RBD) of viral spike protein in complex with ACE2 has been solved. Compared with SARS-CoV, several residue changes in the RBD of SARS-COV2 spike stabilize two virus-binding hotspots at the RBDACE2 interface, explaining why SARS-CoV-2 RBD has a higher ACE2-binding affinity than that of SARS-CoV. It is known that the spike protein of coronaviruses facilitates viral entry into target cells. Specifically, recombinant ACE2-Ig was shown to bind SARS-CoV2 RBD with a high affinity and displayed neutralizing effect of SARS-CoV-2 spike pseudotyped virus. As the attachment of spike to ACE2 is a critical step of viral infection, any agent that blocks viral attachment, such as neutralizing antibodies or competitive entry inhibitors, can be applied to prevent viral infection during disease progression.

SARS-CoV2 virus plaque assay is the gold standard assay for the identification of neutralizing antibodies and antiviral agents. This assay is a quantitative method of measuring infectious SARS-CoV-2 by quantifying the plaques formed in the monolayer cell culture upon infection with serial dilutions of a virus specimen. To work with plaque assay, a confluent monolayer of susceptible cells is infected with serial dilutions of SARS-CoV2 and the infectious virus titers are measured in plaque-forming units (PFU). As a single plaque represents the presence of an infectious SARS-CoV2 virion, such plaque assay can be conducted only in laboratories of biosafety level 3 (BSL-3). Although the plaque assay is the gold standard assay for determining viral titer of SARS-CoV2, the safety regulation in the BSL-3 largely limits accessibility of general researchers in fields of life sciences.

However, the above-mentioned known detection methods require a long detection time, ranging from 3 to 4 hours to 5 days, which will cause a huge burden for research units that recently require a lot of manpower and time to detect coronavirus.

In order to solve the above-mentioned problems, those skilled in the art urgently need to develop novel and effective method and platform for detecting binding of a spike protein receptor binding domain of coronavirus in a specimen with human angiotensin-converting enzyme II for the benefit of a large group of people in need thereof.

### SUMMARY OF THE INVENTION

A primary objective of the present invention is to provide a method for enhancing detection activity of an interaction between a spike protein receptor binding domain of coronavirus from a specimen and a human angiotensin-converting enzyme II (hACE2), comprising the following steps: (a) simultaneously ligating a first DNA fragment of a cleavable luciferase to a DNA fragment of the spike protein receptor binding domain of coronavirus from the specimen, and simultaneously ligating a second DNA fragment of the cleavable luciferase to a DNA fragment of the human angiotensin-converting enzyme II, to form a recombinant plasmid; (b) transforming the recombinant plasmid obtained in step (a) into a cell to express the spike protein receptor binding domain of coronavirus and the human angiotensin-converting enzyme II; and (c) detecting the interaction between the spike protein receptor binding domain of coronavirus and the human angiotensin-converting enzyme II in the cell by detecting a luminescence signal using fluorescence staining; wherein the cleavable luciferase comprises a first subunit and a second subunit, and the spike protein receptor binding domain of coronavirus from the specimen is ligated with the first subunit without a linker.

According to an embodiment of the present invention, the coronavirus is severe acute respiratory syndrome coronavirus 2 (SARS-CoV2).

According to an embodiment of the present invention, the cell is a prokaryotic cell or a mammalian cell.

According to an embodiment of the present invention, the mammalian cell is a HeLa cell.

According to an embodiment of the present invention, in step (b), the human angiotensin-converting enzyme II is expressed in an extracellular domain of the cell.

According to an embodiment of the present invention, the cleavable luciferase is a NanoLuc luciferase.

According to an embodiment of the present invention, in step (b), the recombinant plasmid expresses the spike protein receptor binding domain of coronavirus in the cell, producing a recombinant protein ligated with the first subunit of the cleavable luciferase, and the recombinant protein is used as a ligand for detection.

According to an embodiment of the present invention, the intensity of the luminescence signal is dose-dependent with amounts of the ligand for detection and amounts of the cell expressing the human angiotensin-converting enzyme II.

According to an embodiment of the present invention, detection time of the method is less than 20 minutes.

According to an embodiment of the present invention, the method is used to screen a drug for treating coronavirus infection.

According to an embodiment of the present invention, the human angiotensin-converting enzyme II is ligated with the second subunit.

Another objective of the present invention is to provide a platform for enhancing detection activity of an interaction between a spike protein receptor binding domain of coronavirus from a specimen and a human angiotensin-converting enzyme II (hACE2) in a cell, which is established by the method according to claim 1, the platform comprising a cleavable luciferase, wherein the cleavable luciferase comprises a first subunit and a second subunit, and the spike protein receptor binding domain of coronavirus from the specimen is ligated with the first subunit without a linker.

According to an embodiment of the present invention, the coronavirus is severe acute respiratory syndrome coronavirus 2 (SARS-CoV2).

According to an embodiment of the present invention, the cell is a prokaryotic cell or a mammalian cell.

According to an embodiment of the present invention, the mammalian cell is a HeLa cell.

According to an embodiment of the present invention, the human angiotensin-converting enzyme II is expressed in an extracellular domain of the cell.

According to an embodiment of the present invention, the cleavable luciferase is a NanoLuc luciferase.

According to an embodiment of the present invention, detection time of the platform is less than 20 minutes.

According to an embodiment of the present invention, the platform is used to screen a drug for treating coronavirus infection.

According to an embodiment of the present invention, the human angiotensin-converting enzyme II is ligated with the second subunit.

In summary, the effect of the method and the platform for enhancing detection activity of an interaction between a spike protein receptor binding domain of coronavirus from a specimen and a human angiotensin-converting enzyme II is using a cleavable luciferase as a report test for the combination of a spike protein receptor binding domain of coronavirus (such as novel coronavirus) and angiotensin-converting enzyme II. Screening is carried out at the cellular level. The strength of the drug's influence on the interaction between the two molecules can be judged by the strength of the luminescence signal. The detection time can be completed within 20 minutes. In particular, a quick and robust assay is designed to detect the attachment of virus spike to the ACE2 receptor. With the application of NanoBiT technology, the attachment of RBD to ACE2 receptor is detected in just 10 minutes. This RBD-ACE2 attachment assay was firstly applied for the screening of a plurality of drug candidates for treating coronavirus infection.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1A is a schematic diagram of RBD attachment detection by the luciferase in the present invention.

FIG. 1B is a cell staining diagram of RBD attachment detection by the luciferase in the present invention, in which DAPI is 4′,6-diamidino-2-phenylindole, which is a fluorescent dye that can bind strongly to DNA and is used for fluorescent microscopy observation.

FIG. 1C is a schematic diagram of RBD attachment detection by the luciferase in the present invention.

FIG. 1D is a data diagram of the detection of RBD attachment by the luciferase in the present invention.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

As used herein, the term “treating” or “treatment” refers to alleviating, reducing, ameliorating, relieving or controlling one or more clinical signs of a disease or disorder, and lowering, stopping, or reversing the progression of severity regarding the condition or symptom being treated.

As used herein, the term “seamless cloned” refers to compared with the traditional restriction enzyme digestion and ligation cloning, the specific DNA sequence recognized by the restriction enzyme can be completely eliminated before and after the cloning fragment.

In the following embodiments, a novel coronavirus (severe acute respiratory syndrome coronavirus 2, SARS-CoV2) is selected as an example of the coronavirus, and HeLa cells are selected as an example of the cell, but not representing the scope of the claimed invention is limited to this.

As used herein, the term “a first DNA fragment of a cleavable luciferase” refers to the nucleotide sequence of Large BiT (LgBiT) of the NanoLuc luciferase, and the spike protein receptor binding domain of coronavirus from the specimen is ligated with the LgBiT of the NanoLuc luciferase.

As used herein, the term “a second DNA fragment of the cleavable luciferase” refers to the nucleotide sequence of SmBiT of the NanoLuc luciferase, and the human angiotensin-converting enzyme II is ligated with the SmBiT of the NanoLuc luciferase.

As used herein, the terms “first subunit” and “LgBiT” can be used interchangeably.

As used herein, the terms “second subunit” and “SmBiT” can be used interchangeably.

### EXAMPLE 1

### Evaluation of Effect Regarding Method and Platform for Detecting Coronavirus of Present Invention by Receptor Binding Domain (RBD) Attachment Detection

### 1.1 Molecular Cloning and Cell Culture

SARS-CoV-2 S gene (original (catalog number: MC_0101080) and E. coli optimized (catalog number: MC_0101082)) (see https://www.molecularcloud.org/plasmid/pUC57-2019-nCoV-SE.-coli/MC-0101082. html) sequences were acquired from GenScript. Human ACE2 coding gene (NCBI accession number: NM_021804) was obtained from Addgene (Plasmid #1786). To produce a recombinant Spike-RBD-LgBiT ligand, E. coli optimized Spike-RBD domain sequence (obtained from the 331-521 peptide sequence in the complete sequence of E. coli optimized S, the nucleotide sequence is 993-1569, that is: SEQ ID NO: 1) was cloned into pET28a expression vector (Merck, catalog number: 69864) through NcoI and XhoI, Ala-Gly-LgBiT (large subunit of NanoLuc, see https://promega.media/-/media/files/promega-worldwide/europe/promega-germany/16 02_seminartour/brochure_nanobit_2016_engl.pdf?la=de-de, in which the NCBI accession number of the NanoLuc sequence is JQ437370) (coding sequence from Promega, obtained from Promega pBiT1.1-N[TK/LgBiT] vector sequence 815-1291) coding sequence was then incorporated through XhoI (remain in both ends). To ectopically express SmBiT-hACE2 (SmBiT represents small subunit of NanoLuc; hACE2 represents human angiotensin-converting enzyme II) in mammalian cells, full-length hACE2 gene (NCBI accession number: NM_021804.3) was subcloned into a EF-1α promoter-driven mammalian expression vector (from Addgene #140532, see Aisha Yesbolatova et al., (2020), Nat. Commun., 11(1):5701, PMID: 33177522, two ends are PiggyBac transposon inverted repeat sequences, Piggybac left (5′) inverted repeat is SEQ ID NO: 2, Piggybac right (3′)inverted repeat is SEQ ID NO: 3) (which flanked with PiggyBac transposon inverted repeat sequence), SmBiT (VTGYRLFEEIL from Promega)-Ala-Gly-Ala was used site-directed insertion between hACE2 amino acid 17th and 18th residues. To utilize luciferase as reporter in pseudovirus assay, Nluc (NanoLuc, from Promega, Genebank: AFI792290.1)-Gly-Ser-Gly-T2A sequence was amplified and seamless cloned into the upstream of RFP coding sequence in a lentiviral vector (pLAS2w.RFP-C.Ppuro, acquired from RNAicore, Academia Sinica) by in-fusion cloning (Takara).

All cells involved in this research were regularly maintained in DMEM complete medium (Corning, with 10% FBS and 1/100 of Penicillin-Streptomycin solution) and incubated in 37° C. humidified incubator with 5% CO₂. To generate SmBiT-hACE2 expressing cells (HeLa), HeLa cancer cells were co-transfected plasmids containing SmBiT-hACE2 construct mentioned above and PiggyBac transposase. After 48 hr incubation, transfected cells were under hygromycin selection one week and split into 96 well plate in order to get single cell clones. Single cell clones were then expanded and confirmed hACE2 expression by immunofluorescence staining.

### 1.2 Indirect Immunofluorescence Staining

For examining SmBiT-hACE2 expression in single clones and the binding capability with SARS-CoV-2-Spike protein, SmBiT-hACE2 expressing cells were fixed with 4% formaldehyde 10 minutes, and firstly incubated with anti-ACE2 antibody (Novus SN0754 clone, 1:500 dilution) and CoV-2-Spike-S1-hFc recombinant protein (CoV-2-Spike-S1 subunit: YP_009724390.1 (Val16-Arg685)) (Sino Biologicals, 120 ng per coverslip sample) 1 hour at room temperature. Anti-rabbit Alexa Fluor 594 and antihuman Alexa Fluor 488 secondary antibodies were then labeled for imaging by fluorescent microscopy. Images were taken by using Leica DMI6000 microscope with HCX PL FL 63×/1.4 NA oil objective len and Andor Neo sCMOS camera, which were all processed by MetaMorph software (Molecular Devices).

### 1.3 Receptor Binding Domain (RBD) Attachment Assay

SmBiT-hACE2 cells were seeded into 96-well white plate and incubated overnight prior to attachment assay. For recombinant proteins (Spike RBD-His, Spike S1-hFc, full-length spike trimer) (CoV2 Spike RBD subunit: YP_009724390.1 (Arg319-Phe541)) competition, cells were under phosphate buffered saline (PBS) washed once and then incubated with designated quantity of recombinant proteins (which were serial diluted in Opti-MEM I reduced serum media) 15 minutes at 37° C., followed by the addition of recombinant RBD-LgBiT ligand (RBD subunit: YP_009724390.1 (Phe330-Phe521)) (250 ng/well). For chemiluminescence measurement, Nano-Glo live cell assay was used according to manufacturer's instruction. Luminescence signal was recorded by microplate reader (BioTek Synergy HTX) at 37° C. with time-lapsed kinetics program.

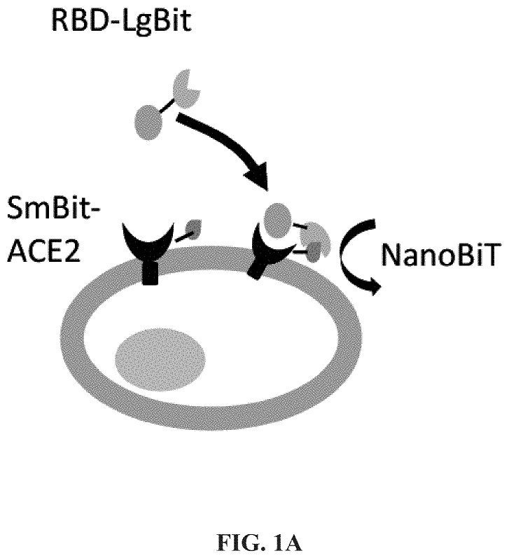

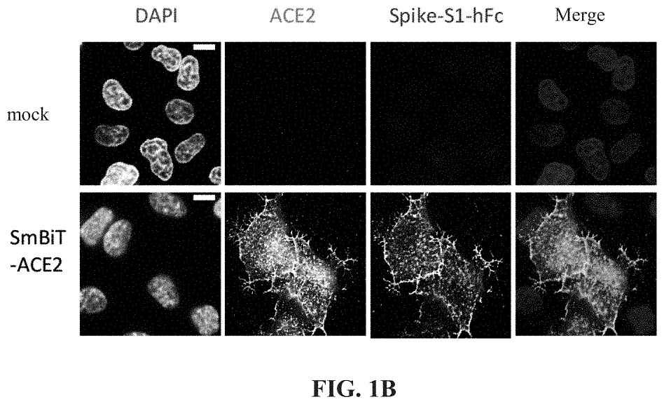

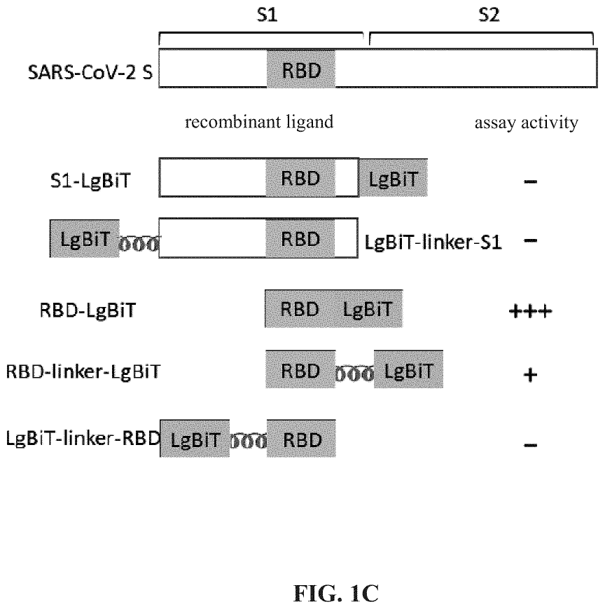

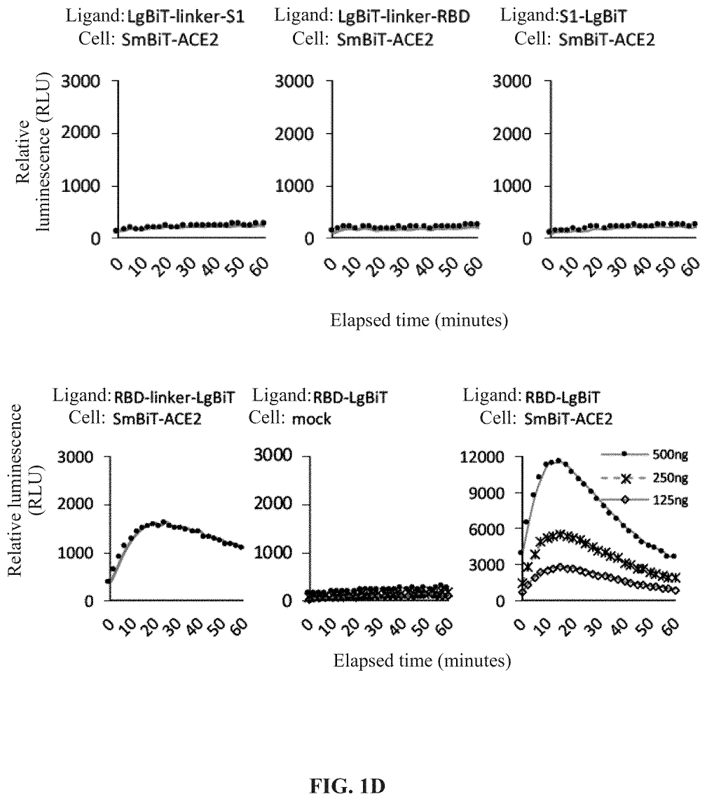

The RBD attachment assay is established with two major components: a stable cell line expressing ACE2 at the cell surface and a recombinant RBD protein with ACE2 binding activity. To monitor successful attachment between RBD and ACE2, the applicant adopted the NanoLuc binary (NanoBiT) technology which allows real-time assays to monitor the dynamics of protein-protein interactions in live cells. Specifically, the NanoBiT system is based on two small subunits of the NanoLuc luciferase, Large BiT (LgBiT) and Small BiT (SmBiT), which expressed as fusions to target proteins of interest, in this case, the RBD and ACE2. Once RBD attaches to the ACE surface receptor, the subunit complementation may occur to reconstitute an active NanoLuc luciferase (FIG. 1A). The applicant has established a stable cell line expressing ACE2 fusion with SmBiT at the N-terminus based on HeLa cells. This stable cell line was treated with hFc-tagged spike S1 protein and only SmBiT-ACE2-expressing cells were labeled by S1-hFc (FIG. 1B). Thus, the N-terminal fusion SmBiT tag of ACE2 did not influence its interaction with S1.

The applicant then generated five different S1/RBD and LgBiT fusion constructs with codon optimization suitable for mass production of recombinant proteins in bacteria (FIG. 1C). The resulted recombinant fusion proteins were incubated with SmBiT-ACE2 cells and assayed for NanoLuc activity. The applicant found that cells incubated with 500 ng of LgBiT-S1 and LgBiT-RBD showed no luciferase activity, same for S1-LgBiT. In striking contrast, adding RBD-LgBiT in SmBiT-ACE2 cells induced a strong and robust luciferase activity (FIG. 1D). The addition of an extra linker (SEQ ID NO: 4) between RBD and LgBiT did not increase the luciferase activity. As the strongest NanoLuc activity was detected with RBD-LgBiT, the applicant then used RBD-LgBiT in following assays. The applicant compared NanoLuc luciferase activity in the experimental setting with mock or SmBiTACE2 cells treated with 125, 250, and 500 ng of RBD-LgBiT. The luciferase activity increased with the amount of RBD-LgBiT ligand in the assay (FIG. 1D). The peak value of luminance was detected at approximately 10 min after adding the substrate in the assay, followed by slow declined of the signal. No luminance signal was detected in mock cells under the same experimental condition. As such, the applicant defined the RBD attachment activity by measuring the peak luminescence signal detected at 10 min of reaction.

In this example, a plurality of recombinant plasmid DNAs express the spike protein receptor binding domain (RBD) of coronavirus in the cell, producing a recombinant protein ligated with a subunit of the luciferase, and the recombinant protein is purified and used as a ligand for detection.

In an example, the specimen used to detect coronavirus can be obtained from a nasopharyngeal or throat wipe, a sputum or a respiratory tract aspirate.

In an example, the intensity of the luminescence signal is dose-dependent with amounts of the ligand for detection and amounts of the cell expressing the human angiotensin-converting enzyme II (ACE2).

The RBD attachment assay of the present invention is a quick and powerful approach that detects the interaction between RBD and ACE2 in just 10 minutes. The entire detection platform operation can be completed in 20 minutes. As this assay does not require BSL-2 laboratory facility, the applicant anticipates such assay can be wildly applied to help identifying novel entry inhibitors, as well as neutralizing antibodies identified from convalescent plasma or experimental animals. In addition, the applicant anticipates this RBD attachment assay can be applied for detecting neutralizing antibodies during vaccine development at early stages. The RBD attachment assay of the present invention can be broadly applied to screen novel viral entry inhibitors and help provide services in a fast and powerful way against COVID-19.

In summary, the effect of the method and the platform for enhancing detection activity of an interaction between a spike protein receptor binding domain of coronavirus from a specimen and a human angiotensin-converting enzyme II is using a cleavable luciferase as a report test for the combination of a spike protein receptor binding domain of coronavirus (such as novel coronavirus) and angiotensin-converting enzyme II. Screening is carried out at the cellular level. The strength of the drug's influence on the interaction between the two molecules can be judged by the strength of the luminescence signal. The detection time can be completed within 20 minutes. In particular, a quick and robust assay is designed to detect the attachment of virus spike to the ACE2 receptor. With the application of NanoBiT technology, the attachment of RBD to ACE2 receptor is detected in just 10 minutes. This RBD-ACE2 attachment assay was firstly applied for the screening of a plurality of drug candidates for treating coronavirus infection.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for enhancing detection activity of an interaction between a spike protein receptor binding domain of coronavirus from a specimen and a human angiotensin-converting enzyme II (hACE2), comprising the following steps:
(a) simultaneously ligating a first DNA fragment of a cleavable luciferase to a DNA fragment of the spike protein receptor binding domain of coronavirus from the specimen, and simultaneously ligating a second DNA fragment of the cleavable luciferase to a DNA fragment of the human angiotensin-converting enzyme II, to form a recombinant plasmid;
(b) transforming the recombinant plasmid obtained in step (a) into a cell to express the spike protein receptor binding domain of coronavirus and the human angiotensin-converting enzyme II; and
(c) detecting the interaction between the spike protein receptor binding domain of coronavirus and the human angiotensin-converting enzyme II in the cell by detecting a luminescence signal using fluorescence staining;
wherein the cleavable luciferase comprises a first subunit and a second subunit, and the spike protein receptor binding domain of coronavirus from the specimen is ligated with the first subunit without a linker.

2. The method according to claim 1, wherein the coronavirus is severe acute respiratory syndrome coronavirus 2 (SARS-CoV2).

3. The method according to claim 1, wherein the cell is a prokaryotic cell or a mammalian cell.

4. The method according to claim 3, wherein the mammalian cell is a HeLa cell.

5. The method according to claim 1, wherein in step (b), the human angiotensin-converting enzyme II is expressed in an extracellular domain of the cell.

6. The method according to claim 1, wherein the cleavable luciferase is a NanoLuc luciferase.

7. The method according to claim 1, wherein in step (b), the recombinant plasmid expresses the spike protein receptor binding domain of coronavirus in the cell, producing a recombinant protein ligated with the first subunit of the cleavable luciferase, and the recombinant protein is used as a ligand for detection.

8. The method according to claim 7, wherein the intensity of the luminescence signal is dose-dependent with amounts of the ligand for detection and amounts of the cell expressing the human angiotensin-converting enzyme II.

9. The method according to claim 1, wherein detection time of the method is less than 20 minutes.

10. The method according to claim 1 is used to screen a drug for treating coronavirus infection.

11. The method according to claim 1, wherein the human angiotensin-converting enzyme II is ligated with the second subunit.

12. A platform for enhancing detection activity of an interaction between a spike protein receptor binding domain of coronavirus from a specimen and a human angiotensin-converting enzyme II (hACE2) in a cell, which is established by the method according to claim 1, the platform comprising a cleavable luciferase, wherein the cleavable luciferase comprises a first subunit and a second subunit, and the spike protein receptor binding domain of coronavirus from the specimen is ligated with the first subunit without a linker.

13. The platform according to claim 12, wherein the coronavirus is severe acute respiratory syndrome coronavirus 2 (SARS-CoV2).

14. The platform according to claim 12, wherein the cell is a prokaryotic cell or a mammalian cell.

15. The platform according to claim 14, wherein the mammalian cell is a HeLa cell.

16. The platform according to claim 12, wherein the human angiotensin-converting enzyme II is expressed in an extracellular domain of the cell.

17. The platform according to claim 12, wherein the cleavable luciferase is a NanoLuc luciferase.

18. The platform according to claim 12, wherein detection time of the platform is less than 20 minutes.

19. The platform according to claim 12 is used to screen a drug for treating coronavirus infection.

20. The platform according to claim 12, wherein the human angiotensin-converting enzyme II is ligated with the second subunit.
